(19) United States
(12) Patent Application Publication
(10) Pub. No.: US 2023/0390159 A1
(43) Pub. Date: Dec. 7, 2023

(54) FOAM-GENERATING DEVICES AND METHODS

(71) Applicant: Moonshot Medical LLC, St Paul, MN (US)

(72) Inventors: Benjamin Arcand, Minneapolis, MN (US); Emily Meyering, Minneapolis, MN (US)

(73) Assignee: Moonshot Medical LLC, St Paul, MN (US)

(21) Appl. No.: 18/131,326

(22) Filed: Apr. 5, 2023

Related U.S. Application Data
(60) Provisional application No. 63/327,783, filed on Apr. 5, 2022.
(60) Provisional application No. 63/415,733, filed on Oct. 13, 2022.
(60) Provisional application No. 63/327,791, filed on Apr. 5, 2022.

(51) Int. Cl. A61J 1/20; A61K 9/12; A61M 5/315; A61M 5/00

(52) U.S. Cl. CPC A61J 1/2089; A61K 9/122; A61M 5/31578; A61M 5/002; A61J 1/2062

(57) ABSTRACT

A kit may include a syringe; and a mixing device. The syringe may have a barrel, a plunger, a tip, and a foamable therapeutic disposed in the barrel. The mixing device may have (A) a syringe body and a mixing tip fluidly coupled to an interior of the syringe body, and a plunger and spring disposed within the syringe body; (B) a mixing channel, a supply channel and a delivery channel; wherein one end of the mixing channel is coupled to the mixing tip and one end of the supply channel includes a connector for removably coupling to the syringe; (C) a three-way valve that is configured to selectively couple an opposite end of the mixing channel, an opposite end of the supply channel and the delivery channel to one or more of the other; and (D) a mixing element disposed in the mixing channel.

### CROSS-REFERENCE TO RELATED APPLICATIONS

This application claims the benefit of U.S. Provisional Application Ser. No. 63/327,783, titled “Mixing Syringes and Methods for Their Use” filed on Apr. 5, 2022; U.S. Provisional Application Ser. No. 63/327,791, titled, “Sclerotherapy Formulations,” filed on Apr. 5, 2022; and U.S. Provisional Application Ser. No. 63/415,733, titled “Foam-Generating Device and Method,” filed on Oct. 13, 2022. This application herein incorporates by reference the foregoing applications.

### TECHNICAL FIELD

Various implementations relate generally to devices and methods for producing foam for use in medical and/or veterinary applications. Various implementations relate generally to mixing two components of a medical or diagnostic agent (e.g., two liquids, a liquid and a gas, a liquid and a solid). In some implementations, the mixing may occur immediately prior to use of the medical or diagnostic agent.

### BACKGROUND

In some implementations, it may be advantageous to prepare a foam for use in medical and/or veterinary applications. The foam may be generated immediately prior to use, and it may be generated from one or more components. The components may include liquids, gases, solids, or some combination thereof.

### SUMMARY

A method of making and providing a therapeutic foam may include providing (i) a syringe and (ii) a mixing device. The syringe may have a barrel, a plunger, a tip, and a foamable therapeutic disposed in the barrel. The mixing device may have (A) a syringe body and a mixing tip fluidly coupled to an interior of the syringe body, and a plunger and spring disposed within the syringe body; (B) a mixing channel, a supply channel and a delivery channel; wherein one end of the mixing channel is coupled to the mixing tip and one end of the supply channel includes a connector for removably coupling to the syringe; (C) a three-way valve that is configured to selectively couple an opposite end of the mixing channel, an opposite end of the supply channel and the delivery channel to one or more of the others; and (D) a mixing element disposed in the mixing channel. The method may further include coupling the syringe to the connector; actuating the three-way valve to couple the supply channel to the mixing channel, but not to the delivery channel; plunging the plunger of the syringe to force foamable therapeutic through the supply channel, mixing channel and mixing element and into the mixing device, thereby causing the spring to be compressed; releasing force on the plunger of the syringe to allow the spring to force the foamable therapeutic back through the mixing channel, mixing element and supply channel and into the syringe, thereby creating a foamed therapeutic; actuating the three-way valve to couple the supply channel to the delivery channel, but not to the mixing channel; and plunging the plunger to dispense the foamed therapeutic.

The method may further include repeating the plunging and releasing steps one or more times. In some implementations, the mixing element includes a mesh screen characterized by apertures of between about 100 μm and 500 μm; in some implementations, the mixing element includes a mesh screen characterized by apertures of between about 10 μm and 25 μm; in some implementations, the mixing element includes a sintered or porous material.

A kit may include a syringe; and a mixing device. The syringe may have a barrel, a plunger, a tip, and a foamable therapeutic disposed in the barrel. The mixing device may have (A) a syringe body and a mixing tip fluidly coupled to an interior of the syringe body, and a plunger and spring disposed within the syringe body; (B) a mixing channel, a supply channel and a delivery channel; wherein one end of the mixing channel is coupled to the mixing tip and one end of the supply channel includes a connector for removably coupling to the syringe; (C) a three-way valve that is configured to selectively couple an opposite end of the mixing channel, an opposite end of the supply channel and the delivery channel to one or more of the other; and (D) a mixing element disposed in the mixing channel. The kit may be configured to allow (x) the syringe to be removably coupled to the connector; (y) the three-way valve to be actuated to couple the supply channel to the mixing channel, but not to the delivery channel; (z) the plunger to be plunged to force foamable therapeutic through the supply channel, mixing channel and mixing element and into the mixing device, thereby causing the spring to be compressed; (aa) the plunger to be released, to allow the spring to force the foamable therapeutic back through the mixing channel, mixing element and supply channel and into the syringe, thereby creating a foamed therapeutic; (bb) the three-way valve to be actuated to couple the supply channel to the delivery channel, but not to the mixing channel; and (cc) the plunger to be plunged to dispense the foamed therapeutic.

A method of making a therapeutic foam may include providing a syringe having a plunger; a housing coupled to the syringe; and a container. The housing may have a first inlet port that couples to the syringe, a second inlet port having a first needle and a second needle, an outlet port, a mixing chamber, a first check valve that permits fluid communication from the second inlet port to the mixing chamber but not from the mixing chamber to the second inlet port, a second check valve that permits fluid communication from the mixing chamber to the outlet port but not from the outlet port to the mixing chamber, and at least one screen disposed between the first check valve and the mixing chamber, or between the second check valve and the mixing chamber, or between the first inlet port and the mixing chamber. The container may include a vessel having an opening on one end that is sealed with a pierceable membrane. The vessel may have a biologically compatible gas and a therapeutic agent that are capable of being combined to form a foam. The method of making a therapeutic foam may further include coupling the container to the syringe by piercing the pierceable membrane with the second inlet port, such that the first needle extends beyond the therapeutic agent into a region containing the biologically compatible gas and the second needle extends into the therapeutic agent; deplunging the syringe to draw biologically compatible gas and therapeutic agent from the container into the mixing chamber and syringe to thereby form a therapeutic foam; and plunging the syringe to expel the therapeutic foam from the housing.

In some implementations, the first needle and second needle comprise a dual-lumen needle; the second needle may be concentrically disposed around the first needle; and the first needle may extend beyond the second needle. The therapeutic agent may be in liquid form in the container. The pierceable membrane may be a self-healing pierceable membrane. The container may be made of glass. The container may include a coated material that inhibits diffusion of gases into or out of the container. At least one screen may include a mesh having openings of about 25 μm. At least one screen may include a mesh having openings of about 10 μm.

The container may further include a pressure-equalization channel that is fluidly coupled to an expandable pressure-equalization chamber and that is configured to couple to a pressure-equalization passage in the housing. The pressure-equalization passage may be open to an exterior of the housing on one end and include a needle at its opposite end, which needle is configured to pierce the pierceable membrane and couple to the pressure-equalization channel when the container is disposed on the housing. The interior of the expandable pressure-equalization chamber may be isolated from the therapeutic agent and biologically compatible gas in the container. The expandable pressure-equalization chamber may include an expandable balloon structure that is configured to inflate with gas that enters its interior through the one end, the pressure equalization passage and the pressure-equalization channel whenever a negative pressure exists in an interior of the container, such that its inflation and corresponding increase in volume displaces therapeutic agent and biologically compatible gas that has been withdrawn from the container.

A mixing and delivery device may include a syringe, a mixing channel, a seal and a stopcock. The syringe may have a barrel, a plunger, and a tip. The barrel may have a sidewall that, with the plunger, defines an interior space. The interior space may include a first fluid component and be fluidly coupled to a discharge port at the tip. The mixing channel may have a channel wall that is characterized by a thickness, that defines an interior volume and that has an outer surface. The mixing channel may have an inlet end, an outlet end, and a plurality of through-pores disposed through the thickness to fluidly couple the interior volume and a space adjacent and exterior to the mixing channel. The mixing channel may further include a flexible membrane that circumferentially surrounds the outer surface and that is sealed to the outer surface at the inlet end and the outlet end. The interior volume may include a second fluid component. The seal may be disposed at the inlet end to initially separate the first fluid component and the second fluid component. The stopcock may have an inlet, an outlet and a valve, and the inlet may be coupled to the outlet end. The valve may have an open configuration that facilitates fluid coupling of the inlet and outlet and a closed configuration that prevents fluid coupling of the inlet and outlet.

In some implementations, the seal is a sealing membrane that is configured to rupture when the plunger is depressed, thereby allowing the first fluid component and the second fluid component to mix.

The flexible membrane may be configured to distend to facilitate transport of fluid from the interior space and interior volume, through the plurality of through-holes, into a mixing space, when the plunger is depressed. The flexible membrane may have an elasticity which, when the flexible membrane is in a distended state, exerts a force on fluid in the mixing space causing said fluid to be forced back through the through-holes, into the interior volume, when such exerted force exceeds counterbalancing pressure of the fluid.

### BRIEF DESCRIPTION OF THE DRAWINGS

FIG. 1 illustrates an exemplary device for generating a therapeutic foam having a syringe, a housing, and a container.

FIG. 2A illustrates the device of FIG. 1, with the container disposed on the housing.

FIGS. 2B-2D depict the device of FIG. 1, as a plunger of the syringe is deplunged to draw contents of the container into the housing and syringe, thereby forming a therapeutic foam.

FIG. 2E depicts the device of FIG. 1, as the plunger of the syringe is plunged to expel the therapeutic foam from the housing.

FIG. 3A illustrates another exemplary device for generating a therapeutic foam.

FIG. 3B illustrates the device of FIG. 3A, with a needle attached thereto, and with exemplary therapeutic foam that can be produced therefrom.

FIG. 4 illustrates another exemplary device for generating a therapeutic foam.

FIG. 5 depicts an exemplary method for generating a therapeutic foam.

FIG. 6A illustrates another exemplary device for generating a therapeutic foam having a syringe, a housing, a container, and a pressure-equalization system.

FIG. 6B illustrates the device of FIG. 6A, with the container disposed on the housing.

FIG. 6C depicts the device of FIGS. 6A and 6B, as a plunger of the syringe is deplunged to draw contents of the container into the housing and syringe, thereby forming a therapeutic foam and activating the pressure-equalization system.

FIG. 6D illustrates another exemplary pressure-equalization system.

FIG. 6E depicts the pressure-equalization system of FIG. 6D in operation.

FIGS. 7A-7B illustrate aspects of an exemplary mixing device.

FIGS. 8A-8M depict actuation of an exemplary mixing device and corresponding distension and contraction of a flexible membrane in the device.

FIGS. 8N-8Q depict dispensing of a mixture formed in an exemplary mixing device.

FIG. 9 illustrates another exemplary mixing device.

FIG. 10A illustrates an exemplary mixing kit including a syringe and a mixing device.

FIG. 10B depicts the mixing kit after the syringe has been coupled to the mixing device.

FIG. 10C depicts a portion of a mixing operation in which force is applied to a plunger of the syringe to force a foamable therapeutic into the mixing device.

FIG. 10D depicts a portion of a mixing operation in which a spring in the mixing device forces foamable therapeutic through a mixing channel and mixing element and back into the syringe, thereby forming a foam.

FIG. 10E illustrates a therapeutic foam being dispensed.

### DETAILED DESCRIPTION

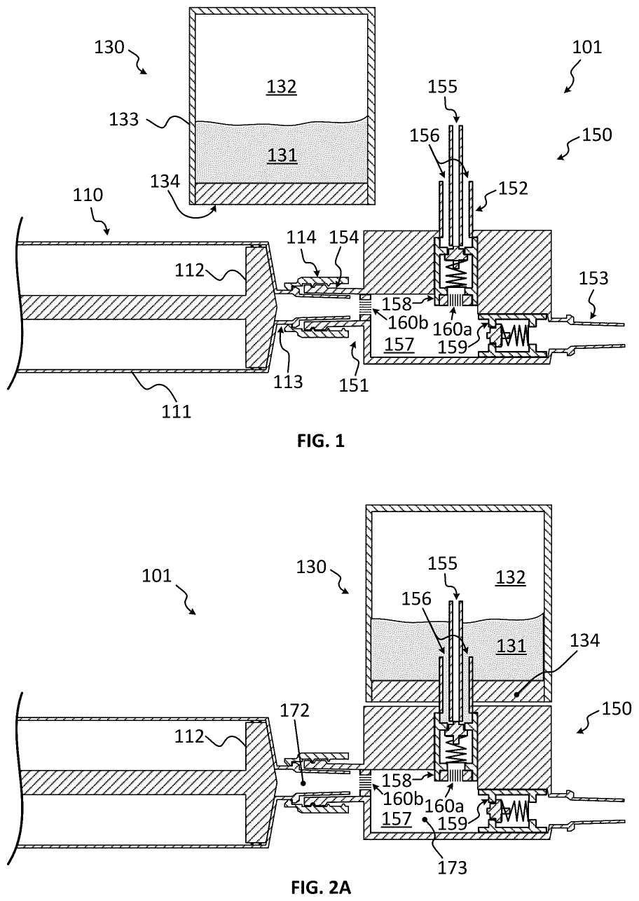

FIG. 1 illustrates a device 101 that can be employed to prepare a therapeutic foam, in some implementations. As shown, the device 101 includes a syringe 110, a container 130 (e.g., a vial or other vessel) and a housing 150. The container 130 may be coupled to the housing 150, and the syringe 110 may be actuated (e.g., deplunged) to withdraw a therapeutic agent 131 (e.g., in liquid form) and a biologically compatible gas 132 from the container 130 and mix the therapeutic agent 131 and biologically compatible gas 132 within the housing 150 and/or syringe 110 to prepare a therapeutic foam. The therapeutic agent may include a drug, a biologic, a vehicle and excipients, or some combination thereof. The syringe 110 may then be actuated (e.g., plunged) to dispense the therapeutic foam.

The syringe 110 may be a standard medical-grade syringe having a barrel 111, a plunger 112 and an outlet port 113. In some implementations, as shown, the syringe 110 includes a Luer lock 114 (or other coupling member) at the outlet port 113 for coupling the syringe 110 to other devices (e.g., a needle, or, as shown, the housing 150). In some implementations, the syringe 110 is a 10 mL, 20 mL, 30 mL or 40 mL syringe; in other implementations, the syringe 110 has another suitable volume.

The container 130 may include a vessel wall 133 that is open on the end; and the open end may be sealed with a membrane 134. In some implementations, the membrane 134 is a pierceable, self-healing membrane that is configured to accommodate a needle for accessing its contents. In some implementations, the vessel wall 133 comprises glass to inhibit diffusion of gases into or out of an interior of the container 130; in other implementations, the vessel wall 133 comprises a coated material that inhibits diffusion of gas; similarly, the membrane 134 may be configured to inhibit diffusion of gases.

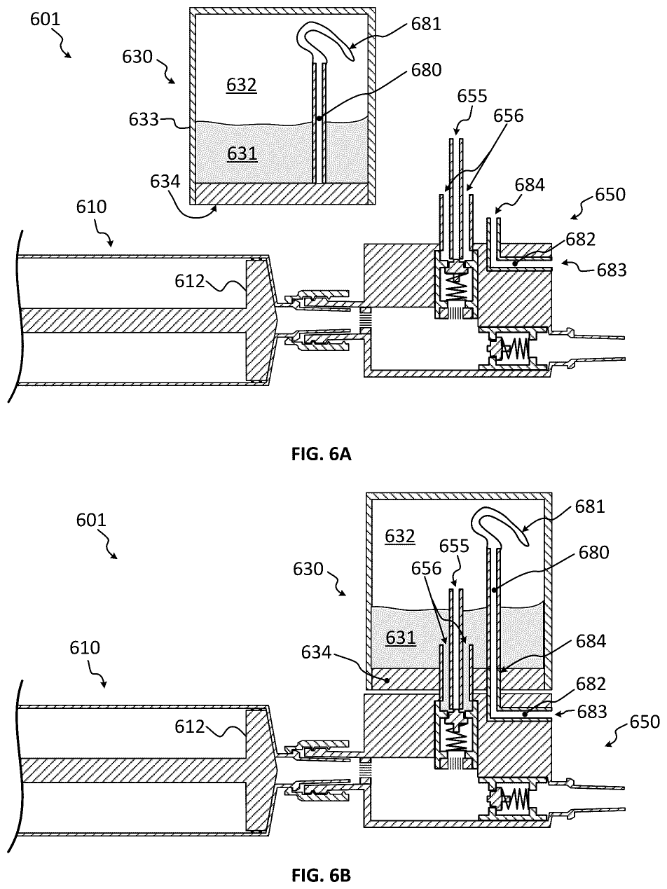

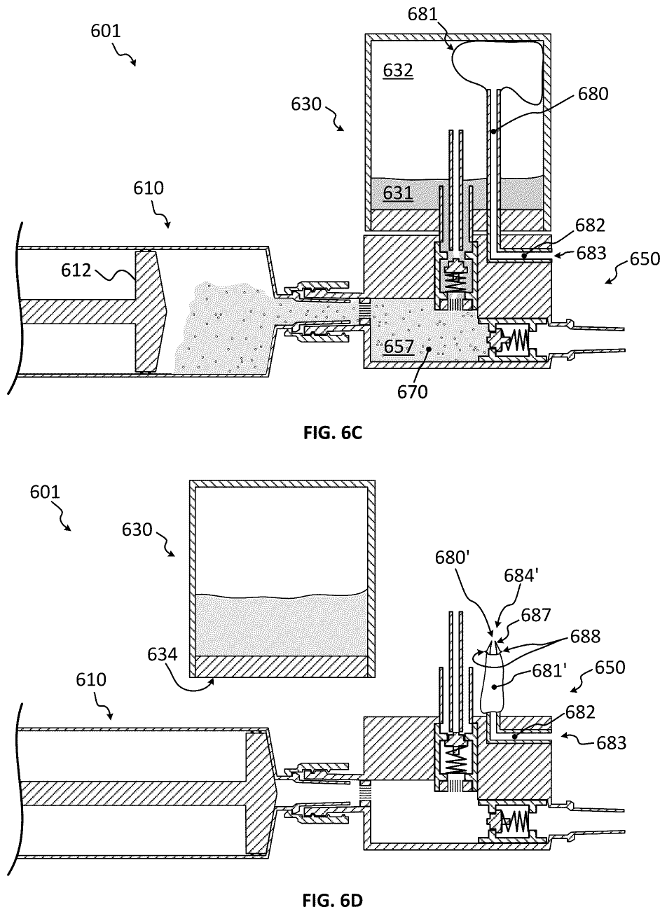

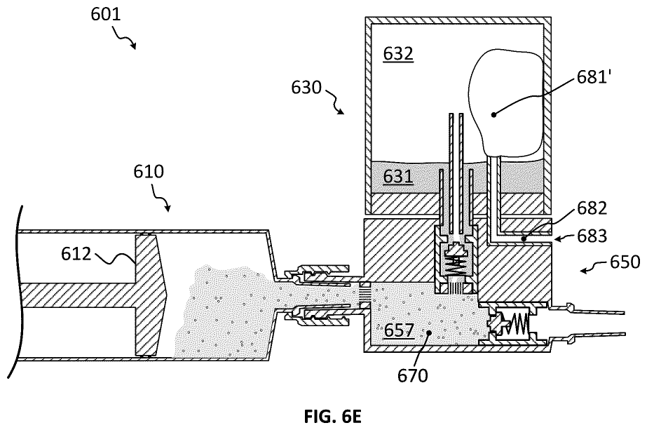

As shown, the housing 150 includes a first inlet port 151, a second inlet port 152 and an outlet port 153. The first inlet port 151 may be configured to couple to the syringe 110, for example, via a corresponding Luer lock fitting 154. The second inlet port 152 may be configured to couple to the container 130. For example, the second inlet port 152 may include a first needle 155 and a second needle 156. In some implementations, as shown, the second needle 156 may be concentrically disposed around the first needle 155; in other implementations, the second needle 156 may be a separate needle, not concentrically disposed around the first needle 155. The first needle 155 may be longer than the second needle 156. Both needles 155 and 156 may be configured to pierce the membrane 134 of the container 130. In some implementations, a needle may have sharpened and/or angled edges (e.g., like needle tip 687 illustrated in FIG. 6D).

The housing 150 may enclose a mixing chamber 157 that can be in fluid communication with the first inlet port 151, the second inlet port 152, and the outlet port 153. In some implementations, as shown, a first check valve 158 is disposed between the mixing chamber 157 and the second inlet port 152—such that fluid communication is permitted from the second inlet port 152 to the mixing chamber 157, but not from the mixing chamber 157 to the second inlet port 152. A second check valve 159 may be disposed between the mixing chamber 157 and the outlet port 153—such that fluid communication is permitted from the mixing chamber 157 to the outlet port 153, but not from the outlet port 153 to the mixing chamber 157. In other implementations, other check valves may be included, or the check valves 158 or 159 may be differently disposed.

A screen 160a may be disposed, as shown, between the first check valve 158 and the mixing chamber 157. In some implementations, the screen 160a expedites formation of a therapeutic foam by facilitating mixing of components (e.g., liquid and gas components) that pass through the screen 160a from the first inlet port 152. In some implementations, the screen 160a comprises a mesh with openings of a particular size, such as, for example, about 10 μm or about 25 As used herein, “about” or “approximately” or “substantially” may mean within 1%, or 5%, or 10%, or 20%, or 50%, or 100% of a nominal value.

In other implementations, mesh openings may have other sizes, such as, for example, between about 25 μm and 100 μm, or between about 0.5 μm and 500 μm. In some mesh-based implementations, “pore density” (e.g., the fraction of an overall space occupied by a mesh that comprises the pore openings (in contrast to space occupied by the material that comprises the mesh itself)) may vary. In still other implementations, a mesh may be replaced by other media, such as, for example, a sintered disc, another sintered element, a porous disc, another porous element, etc.

In some implementations, elements (e.g., screens, meshes, sintered elements, porous elements) may be arranged in-line with each other, such that liquid and gas components are progressively mixed in stages; for example, some implementations may employ multiple screens each comprising differently dimensioned meshes; as another example, some implementations may employ a screen in-line with a sintered element.

In general, pore size and pore density may be adjusted to optimize formation of a therapeutic foam having specific characteristics (e.g., a particular density, a specific proportion of components, a mean air bubble size, a minimum half-life, etc.); moreover, pore size and pore density may be adjusted to optimize (e.g., minimize, in some implementations) back pressure presented by the corresponding porous element.

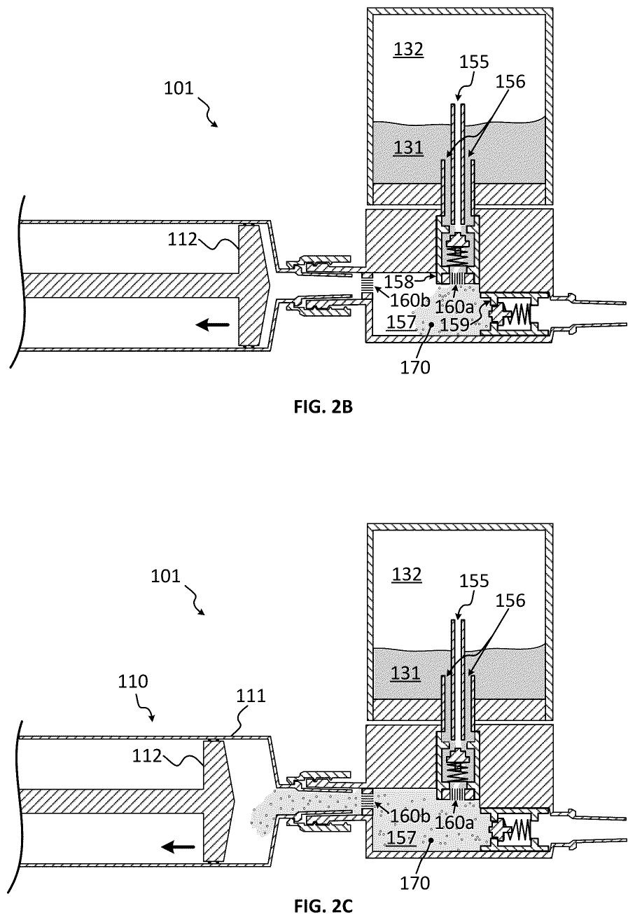

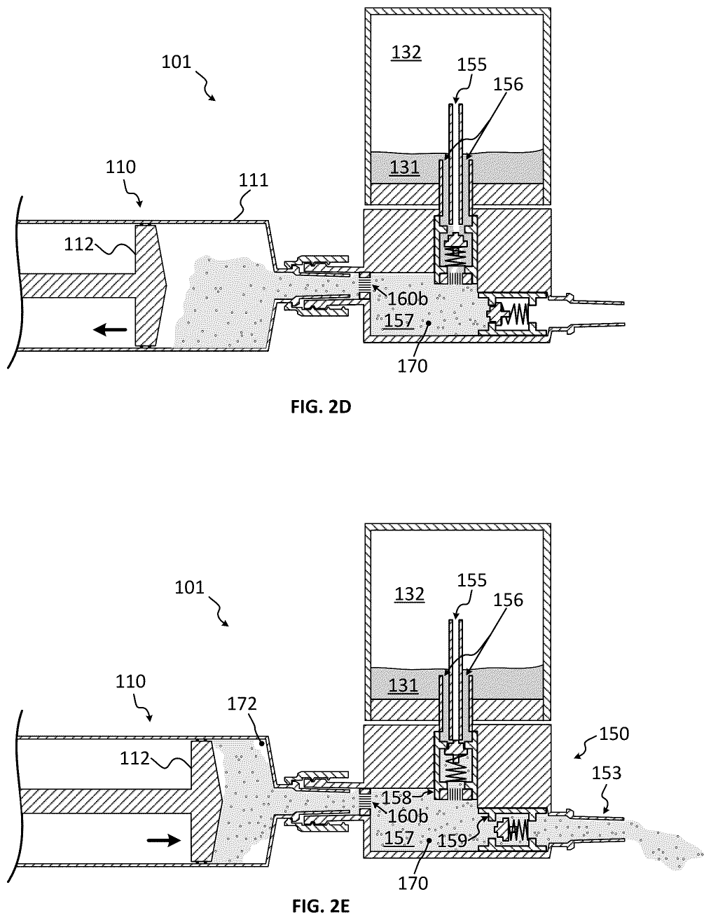

FIG. 2A illustrates the device 101 in a configuration in which the container 130 has been disposed on the housing 150. As shown, the first needle 155 is disposed through the membrane 134, and its opening is in contact with the biologically compatible gas 132 in the container 130; and the second needle 156 is also disposed through the membrane 134, and its opening is in contact with the therapeutic agent 131 in the container 130. In some implementations, as shown, the membrane seals around the needles 155 and 156, such that the gas 132 and agent 131 from within the container 130 only leave the container 130 through lumens of the needles and 155 and 156 (and not by leaking out through any openings between the membrane and the needles 155 and 156).

In the implementation shown, with an opening of the needle 156 in contact with the agent 131, the agent 131 is able to pass into the needle 156, to the check valve 158. The plunger 112 is shown in an initial, fully plunged position, and the mixing chamber 157 is depicted at atmospheric pressure, such that both check valves 158 and 159 remain closed.

FIG. 2B depicts a state in which the plunger 112 has been slightly deplunged, creating a negative pressure within the mixing chamber 157. As depicted, this negative pressure overcomes the holding force of the check valve 158, allowing agent 131 and gas 132 to be drawn through respective needles 156 and 155, through the check valve 158, through the screen 160a, and into the mixing chamber 157. As the agent 131 and gas 132 are drawn through the screen 160a, the gas 132 may aerate the agent 131 and form a foam 170, as depicted.

FIG. 2C depicts a state in which the plunger 112 has been further deplunged, sustaining and/or increasing the negative pressure within the mixing chamber 157 and drawing additional agent 131 and gas 132 through the screen 160a and into the mixing chamber 157, forming additional foam 170. As depicted in some implementations, the foam may be drawn through a second screen 160b, as it is drawn into an interior of the barrel 111 of the syringe 110. As depicted in FIG. 2D, the plunger 112 has been further deplunged, drawing still more agent 131 and gas 132 into the mixing chamber 157, and forming still more foam 170.

FIG. 2E depicts a state in which the plunger 112 has been plunged, thereby creating a positive pressure in the interior of the barrel 111 and in the mixing chamber 157. As depicted, this positive pressure causes the check valve 158 to close and causes the check valve 159 to open, allowing foam 170 to be expelled from the housing 150 via its outlet port 153.

In some implementations, a plunging action of the plunger 112 that causes foam 170 to be plunged through the screen 160b further agitates and aerates the foam 170 by breaking up larger bubbles within the foam 170 into smaller ones and allowing gas 172 within the interior of the barrel 111 to mix with the foam 170.

In some implementations, gas within the housing 150 and the syringe 110 may be controlled. For example, with reference to FIG. 2A, gas 172 in the syringe 110 and gas 173 in the mixing chamber 157 may be biologically compatible gases that are the same as or similar to the gas 132 in the container 130. To maintain such gases 172 and 173 and to keep atmospheric gases outside of the syringe 110 or the housing 150 prior to use, the syringe 110 and housing 150 may be preassembled together and filled with desirable biologically compatible gas; and additional internal seals (not shown) may be provided (e.g., in the outlet port 113 and/or first inlet port 151).

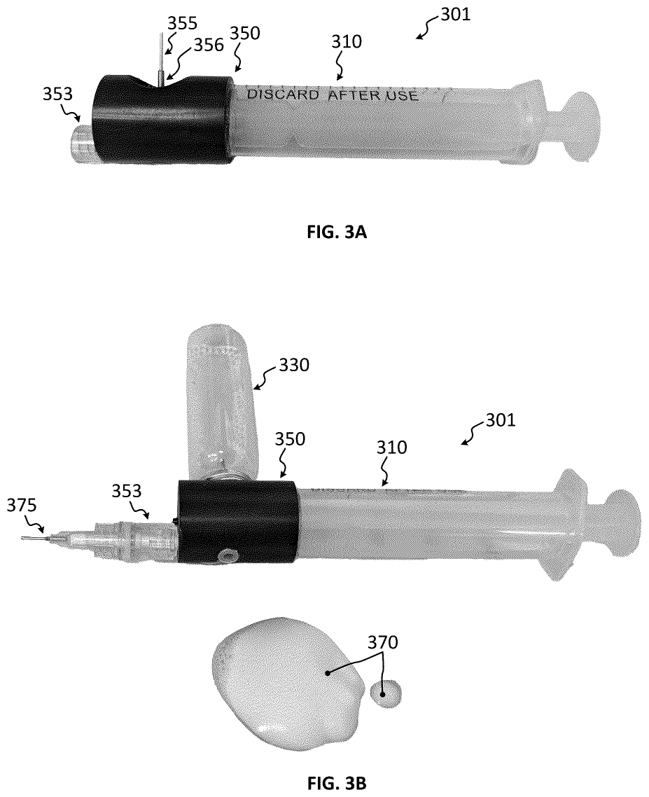

Another implementation of a mixing device 301 is shown in FIG. 3A and has a syringe 310 and a housing 350, with a first needle 355, second needle 356 and an outlet port 353. FIG. 3B illustrates the mixing device 301 with a container 330 disposed on the housing 350 (e.g., with the needles 355 and 356 disposed through a membrane (not shown) of the container 330), and with a needle 375 disposed on the outlet port 353, as well as a quantity of foam 370 produced by the mixing device 301.

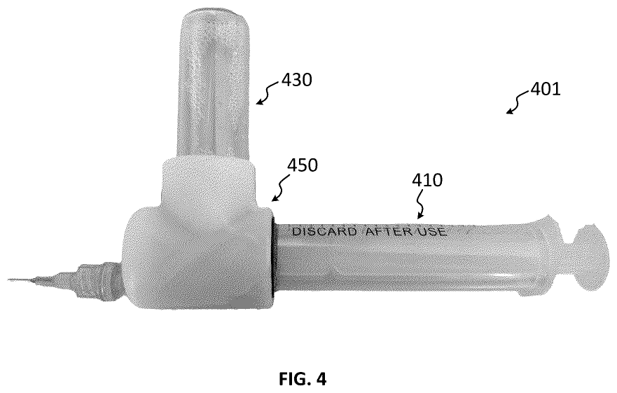

FIG. 4 illustrates another implementation of a mixing device 401 that includes a syringe 410, a container 430 and a housing 450.

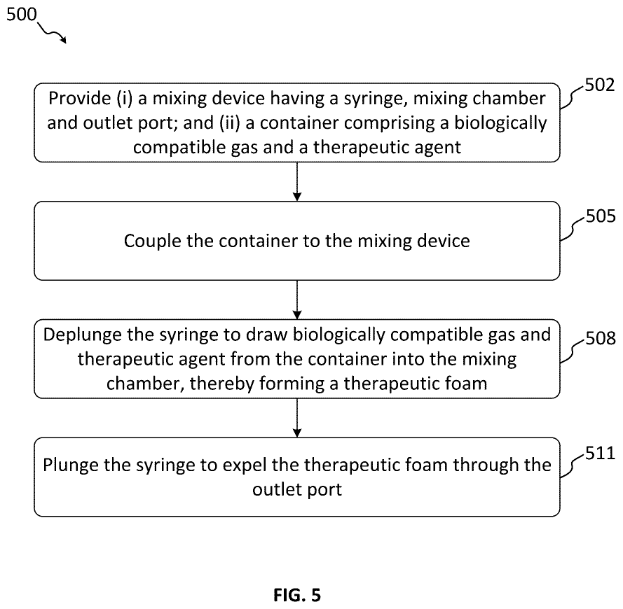

FIG. 5 illustrates an exemplary method 500 for using a mixing device to prepare a therapeutic foam. The method 500 may include providing (502) a mixing device having a syringe, a mixing chamber and an outlet port; and a container comprising a biologically compatible gas and a therapeutic agent. For example, the method 500 could include providing (502) the device 101 shown in and described with reference to FIGS. 1 and 2A-2E, which has a syringe 110, a mixing chamber 157 and an outlet port 153; and further providing a container 130 containing a biologically compatible gas 132 and a therapeutic agent 131.

The method 501 may further include coupling (505) the container to the mixing device. For example, referring to FIG. 2A, the container 130 may be coupled (505) to the device 101. More specifically, the container 130 may be coupled (505) to the device 101 on the housing 150 such that the second inlet port 152 (comprising the first needle 155 and second needle 156) is disposed through the membrane 134 of the container 130. In some implementations, the membrane 134 seals around the second inlet port 152, such that there is substantially no fluid communication between an interior of the container 130 and an exterior of the container 130, except through the second inlet port 152; moreover, the membrane 134 may be self-healing, such that if the container 130 is disengaged from the housing 150 and second inlet port 152, an interior of the container 130 is again sealed from an exterior of the container 130—preventing any biologically compatible gas 132 or therapeutic liquid 131 from escaping from the container 130 and preventing any gas, liquid or solid outside of the container 130 from entering the container 130.

The method 501 may further include deplunging (508) the syringe to draw biologically compatible gas and therapeutic agent from the container into the mixing chamber, thereby forming a therapeutic foam. For example, with reference to FIG. 2B, the plunger 112 may be deplunged (508) (e.g., from a neutral, fully plunged initial position). As depicted, the deplunging (508) action may create a negative pressure in the mixing chamber 157, which may cause the first check valve 158 to open, allowing therapeutic agent 131 to be drawn into the mixing chamber 157 through the second needle 156 and biologically compatible gas 132 to be drawn into the mixing chamber 157 through the first needle 155. As the biologically compatible gas 132 and therapeutic agent 131 are drawn into the mixing chamber 157, they may mix within the first check valve 158, and further mixing may be facilitated by the screen 160a, such that a therapeutic foam 170 is formed in the mixing chamber 157. As the plunger 112 continues to be deplunged (508), additional therapeutic liquid 131 and biologically compatible gas 132 may be drawn into the mixing chamber 157 and into an interior of the barrel 111 of the syringe 110, forming additional therapeutic foam 170. In some implementations, the therapeutic foam 170 is drawn through a screen 160b as it passes from the mixing chamber 157 into the syringe 110.

The method 501 may further include plunging (511) the syringe to expel the therapeutic foam through the outlet port. For example, with reference to FIG. 2E, the plunger 112 may be plunged, creating a positive pressure within the syringe 110 and mixing chamber 157. Such a positive pressure may cause the first check valve 158 to close and the second check valve 159 to open, enabling the therapeutic foam 170 to be expelled through the outlet port 153.

As shown, therapeutic foam 170 that is in the syringe 110 may be forced back through the screen 160b. In some implementations, passage of the therapeutic foam 170 through the screen 160b a second time may further agitate the therapeutic foam 170, causing gas and liquid components to more thoroughly mix and causing larger gas bubbles therein to be broken down into smaller gas bubbles. Passage of the therapeutic foam through the second check valve 159 may have a similar effect of further agitating the foam and causing greater mixing of gas and liquid components and further breakdown of larger gas bubbles into smaller gas bubbles. Moreover, some gas 172 in the syringe 110 may be introduced into the therapeutic foam 170 by the positive pressure and/or by passage of the therapeutic foam 170 and gas 172 through the screen 160b and second check valve 159.

FIG. 6A illustrates another implementation of a mixing device 601 that includes a syringe 610, a housing 650 and a container 630. In the implementation shown, the container 130 includes a pressure-equalization channel 680 and an expandable pressure-equalization chamber 681 interior to the container 630—as well as a vessel wall 633 and membrane 634 that can enclose a therapeutic agent 631 and biologically compatible gas 632. As shown, the housing 650 includes a pressure-equalization passage 682 that fluidly couples an open end 683 (e.g., an end of the passage 682 that is open to the atmosphere) to a needle 684.

As depicted in one implementation in FIG. 6B, when the container 630 is coupled to the housing 650 (e.g., by the needles 655 and 656, which pierce the membrane 634 to fluidly couple with the biologically compatible gas 632 and therapeutic agent 631, respectively, as in other implementations), the pressure-equalization passage 682 may be coupled to the pressure-equalization channel 680 (e.g., by the needle 684 piercing the membrane 634 and aligning with and coupling to the pressure-equalization channel 680). In this manner, an interior of the expandable pressure-equalization chamber 681 can be coupled to the atmosphere (e.g., outside of both the container 630 and the housing 650, via the open end 683 of the pressure-equalization passage 682); moreover, an interior of the expandable pressure-equalization chamber 681 can be isolated from an interior of the container 630 by the wall of the expandable pressure-equalization chamber 681 itself (which, in some implementations, may take the form of a flexible balloon or similar volume-expandable structure)—preventing contamination of the therapeutic agent 631 and the biologically compatible gas 632, in some implementations.

In operation, as depicted in FIG. 6C, when the plunger 612 of the syringe 610 is deplunged—creating a negative pressure within the syringe 610 and within a mixing chamber 657 of the housing 650 and causing the therapeutic agent 631 and biologically compatible gas 632 to be drawn into the mixing chamber 657 and to form a therapeutic compound 670—the resulting negative pressure inside the container 630 (e.g., from the withdrawal of a quantity of the therapeutic agent 631 and the biologically compatible gas 632) can be balanced by an expansion of the expandable pressure-equalization chamber 681. That is, negative pressure inside the container 630 can allow air at atmospheric pressure to enter the open end 683 of the pressure-equalization passage 682 and flow through the pressure-equalization channel 680 and into the expandable pressure-equalization chamber 681, causing its volume to increase to balance the loss in volume associated with the quantity of the therapeutic agent 631 and the biologically compatible gas 632 withdrawn.

In another implementation, as illustrated in FIG. 6D, an expandable pressure-equalization chamber 681′ may be disposed on the housing 650—for example, around a needle 684′ that forms the pressure-equalization channel 680′. The needle 684′ may include a tip 687 with sharpened and/or angled edges to facilitate piercing the membrane 634 on the container 630. As shown in one implementation, the needle 684′ may also include nubs 688 to facilitate passage of the material forming the pressure-equalization chamber 681′ through an opening in the membrane 634 created by the needle 684′ (e.g., by temporarily creating a larger opening in the membrane 634 than the pressure-equalization channel 680′).

In operation, as depicted in FIG. 6E, when the plunger 612 of the syringe 610 is deplunged—creating a negative pressure within the syringe 610 and within a mixing chamber 657 of the housing 650 and causing the therapeutic agent 631 and biologically compatible gas 632 to be drawn into the mixing chamber 657 and to form a therapeutic compound 670—the resulting negative pressure inside the container 630 (e.g., from the withdrawal of a quantity of the therapeutic agent 631 and the biologically compatible gas 632) can be balanced by an expansion of the expandable pressure-equalization chamber 681′. That is, negative pressure inside the container 630 can allow air at atmospheric pressure to enter the open end 683 of the pressure-equalization passage 682 and into the expandable pressure-equalization chamber 681′, causing its volume to increase to balance the loss in volume associated with the quantity of the therapeutic agent 631 and the biologically compatible gas 632 withdrawn.

In implementations such as those just described, the balancing of volumes and pressures can reduce a resistive force exerted through the plunger 612 as it is deplunged. Thus, a user of the syringe 610 may be able to more easily actuate (e.g., deplunge) the syringe 610 to form therapeutic foam 670, relative to implementations without a pressure-equalization system. In some implementations, a machine rather than a human user may actuate the syringe to form a therapeutic foam. In such implementations, the device 601 shown in and described with reference to FIGS. 6A-6C may have the advantage of requiring a linear or near-linear force to actuate the plunger 612.

In other implementations, devices and methods may be employed for mixing two components of a medical or diagnostic agent (e.g., immediately prior to use). In some implementations, a liquid component and a gas component are combined to prepare an agent, for example, for use in a diagnostic or therapeutic procedure in which ultrasound imaging may be required. In other implementations, two liquid components are combined to prepare an agent, for example, for use as a sclerosant, in a sclerotherapy procedure, or for use as a coagulant in a diagnostic or surgical procedure. In other implementations, a liquid component and a solid component are combined to prepare an agent, for example, for use in one of the foregoing applications or in another application.

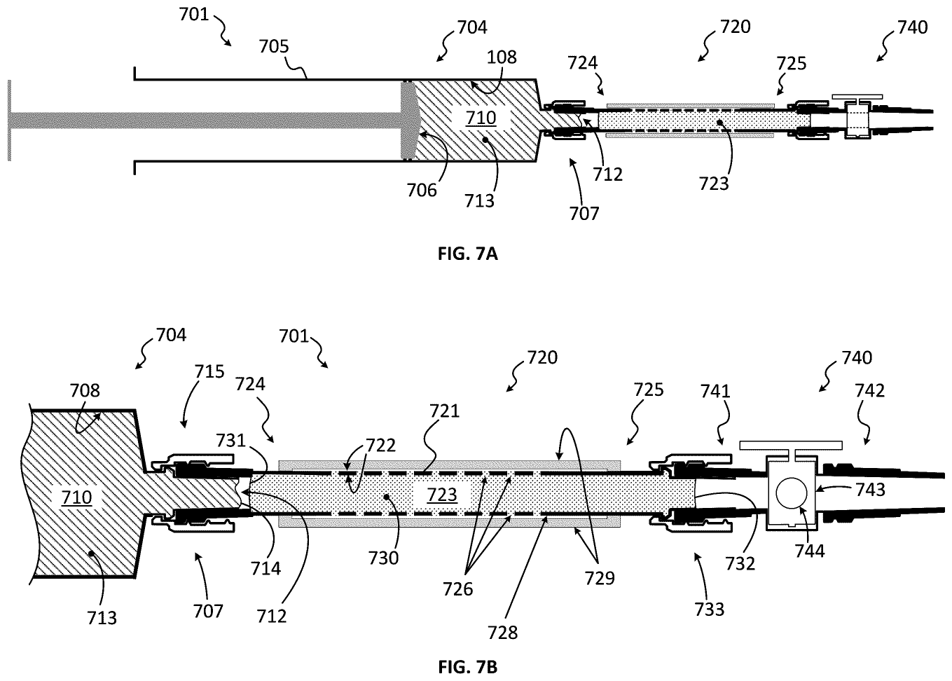

FIG. 7A illustrates an exemplary mixing device 701. As shown, the exemplary mixing device 701 includes a syringe 704, a mixing channel 720, and a stopcock 740. The syringe 704 includes a barrel 705, a plunger 706, and a tip 707. The barrel 705 has a sidewall 708, which, in conjunction with the plunger 706 forms an interior space 710. The interior space 710 is fluidly coupled to a discharge port 712 at the tip 707.

FIG. 7B provides a magnified view of a portion of the exemplary mixing device 701. As shown, the mixing channel 720 has a channel wall 721 that is characterized by a thickness 722, which defines an interior volume 723. The mixing channel 720 has an inlet end 724 and an outlet end 725. Disposed through the thickness 722 are a plurality of though-pores 726, which fluidly couple the interior volume 723 and a space adjacent and exterior to the mixing channel 720. The channel wall 721 has an outer surface 728. Circumferentially surrounding the outer surface 728 is a flexible membrane 729.

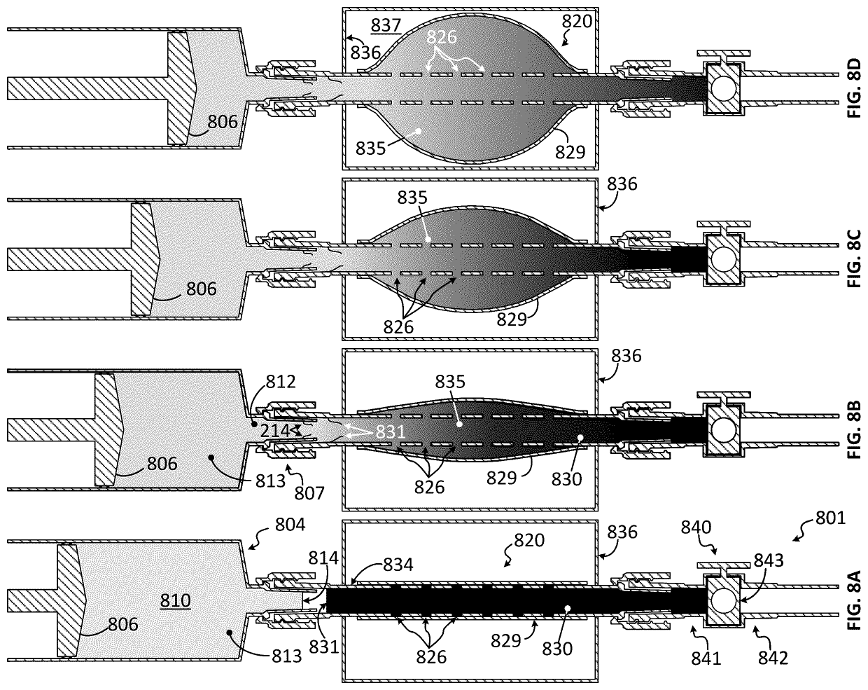

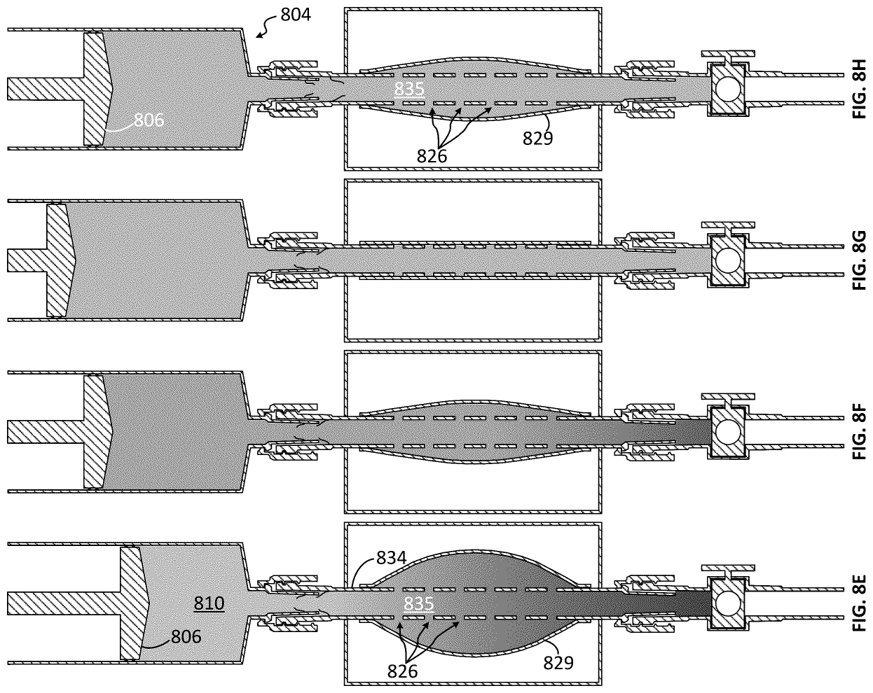

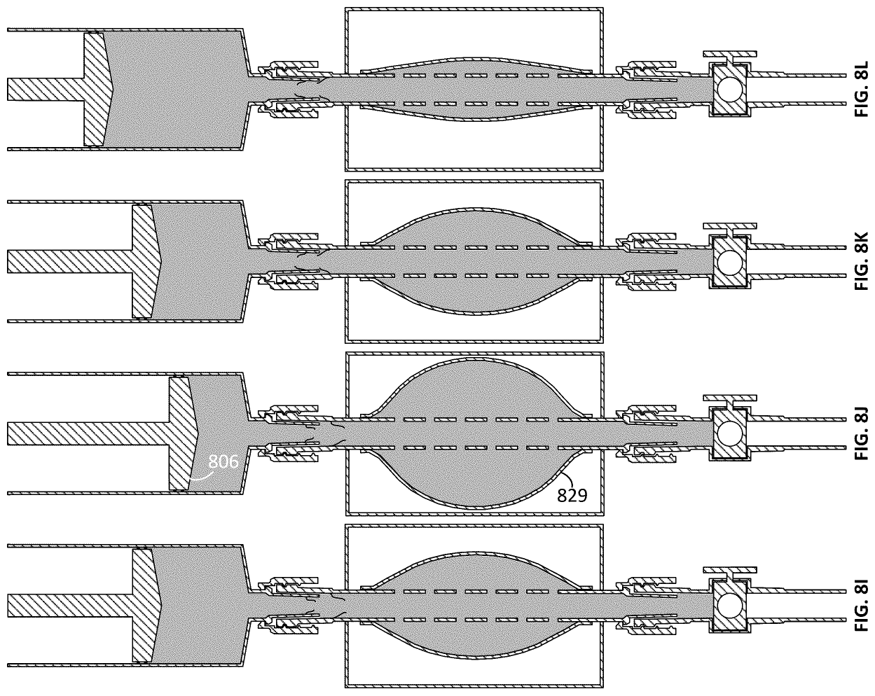

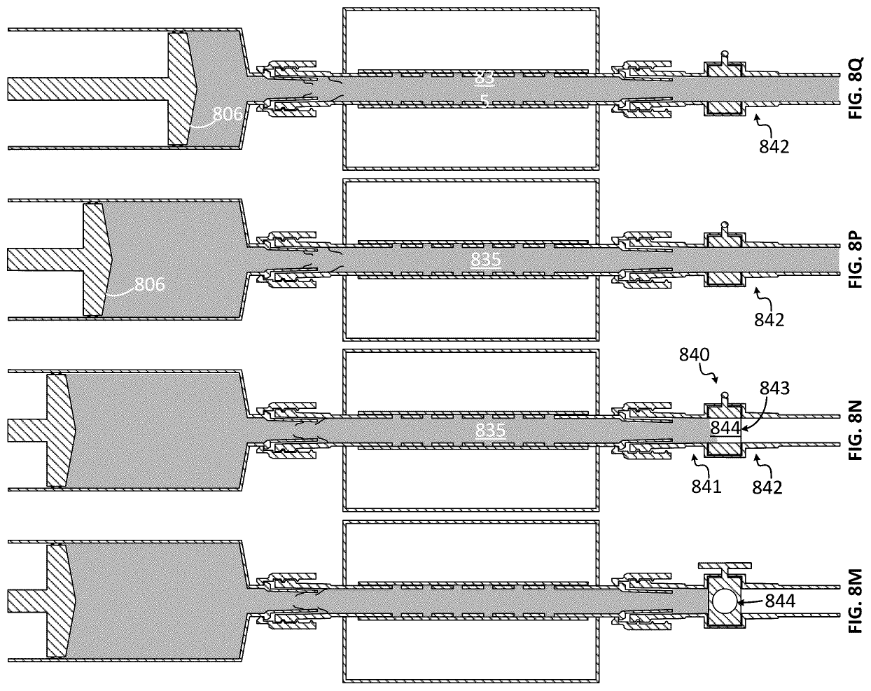

As will be described in greater detail with reference to an implementation illustrated in FIGS. 8A-8M, the flexible membrane 729 may be configured to expand when pressure in the interior volume 723 increases, causing liquid or gas in that interior volume 723 to be forced through the plurality of through-pores 726, into an expandable space bounded by an inner surface of the flexible membrane 729 and the outer surface 728 of the channel wall 721.

The stopcock 740 has an inlet 741, an outlet 742, and a valve 743. The inlet 741 of the stopcock 740 is coupled to the outlet end 725 of the mixing channel 720. The valve 743 has an open configuration that facilitates fluid coupling of the inlet 741 and outlet 742; and a closed configuration that prevents fluid coupling of the inlet 741 and outlet 742. In some implementations, the valve 743 is a cylinder with a transverse hole 744 disposed therethrough. In the open configuration (not shown in FIG. 7B), the transverse hole 744 is aligned with a longitudinal axis of a channel that couples the inlet 741 and the outlet 742, thereby allowing fluid communication between the inlet 741 and the outlet 742; in the closed configuration (shown in FIG. 7B), the transverse hole is aligned perpendicular to the longitudinal axis, thereby preventing fluid communication between the inlet 741 and the outlet 742.

As shown in one exemplary configuration, the syringe 704 may be coupled to the mixing channel 720 with a coupling 715, such as a Luer taper fitting. The mixing channel 720 may be coupled to the stopcock 740 with a similar coupling 733, such as another Luer taper fitting. Other styles of couplings 715 and 733 are possible, such as, for example, threaded couplings, press-fit couplings, etc. In some implementations, various components may be co-molded or an adhesive weld may be used to join various components.

In operation, the mixing device 701 may be employed to mix a first component 713 with a second component 730 prior to the mixture being dispensed from the mixing device 701. In some implementations, the first component 713 is a fluid component, and the second component 730 is a second liquid component. In other implementations, the first component 713 is a fluid component and the second component 730 is a gaseous component. In other implementations, the first component 713 is a fluid component and the second component 730 is a solid component.

As illustrated in FIG. 7B, in an initial configuration, the first component 713 may be disposed in the interior space 710, and the second component 730 may be disposed in the interior volume 723. (In other implementations (not shown), the first component 713 may be disposed in the interior volume 730, and the second component 730 may be disposed in the interior space 710.) A sealing membrane 714 disposed in the tip 707 may initially contain the first component 713 in the interior space 710. Optionally, in some implementations, a second sealing membrane 731 and/or third sealing membrane 732 may contain the second component 730 within the interior volume 723. In other implementations, the second sealing membrane 731 and third sealing membrane 732 are not present. Instead, the stop cock 740 may contain the second component 730 at the outlet end 725 of the mixing channel 720; and the sealing membrane 714 may initially separate the first component 713 and the second component 730. However configured, the sealing membrane 714 and the optional second sealing membrane 731 can prevent the first component 713 and second component 730 from mixing in an initial configuration.

The sealing membrane 714 (and optional second sealing membrane 731 and/or third sealing membrane 732) may be configured to rupture when a pressure impinging thereon exceeds some threshold point. In such implementations, the sealing membrane(s) 714, 731, and/or 732 may contain and separate the first component 713 and second component 730 in an initial configuration (e.g., during shipment and preparation for use of the mixing device 701). When pressure against the sealing membrane 714 (e.g., as the plunger 706 of the mixing device is actuated), the sealing membrane 714 may rupture, allowing the first component 713 to be forced from the interior space 710, through the discharge port 712, into the mixing channel 720. Similarly, if present, the second sealing membrane 731 may also be configured to easily rupture, such that when the plunger 706 is actuated, the first component 713 and second component 730 are able to mix in the mixing channel 720.

Turning to FIGS. 8A-8M, operation of an exemplary mixing device 801 is now described. As shown in FIG. 8A, the exemplary mixing device 801 includes a syringe 804, a mixing channel 820, and a stopcock 840. The mixing channel 820 includes a lumen 834 having a plurality of through-pores 826 about its circumference and length, and a flexible membrane 829 that circumferentially surrounds the lumen 834.

In an initial configuration, a plunger 806 in the syringe 804 has not been actuated; and a first sealing membrane 814 contains a first component 813 in an interior space 810 of the syringe 804. A second component 830 is contained in the mixing channel 820, specifically, as shown, by a second sealing membrane 831 and the stopcock 840, whose valve 843 is closed, preventing fluid communication between an inlet 841 and outlet 842.

FIG. 8B depicts a configuration in which the plunger 806 has been actuated (e.g., by a user partially depressing the plunger 806). Pressure exerted by the plunger 806 on the first component 813 causes the first component 813 to impinge on and rupture both the sealing membrane 814 and second sealing membrane 831, enabling the first component 813 and second component 830 to begin mixing to create a mixture 835. Moreover, the pressure of the resulting mixture 835 forces that mixture 835 through the through-pores 826 and causes the flexible membrane 829 to begin distending or expanding.

As depicted in FIGS. 8C-8D, continued actuation of the plunger 806 forces more of the mixture 835 through the through-pores 826, causing further distension or expansion of the flexible membrane 829. In some implementations, as the mixture is forced through the through-pores 826, additional mixing occurs; and the mixture 835 formed from the mixing of the first component 813 and the second component 830 becomes more homogenous.

In some implementations, as shown in FIGS. 8A-8M, a housing 836 surrounds the mixing channel 820, including its flexible membrane 829. The housing 836 may provide an air-tight seal around the mixing channel 820, such that as the flexible membrane 829 expands, pressure increases inside an air space 837 (see FIG. 8D) that is defined by the housing 836 and the flexible membrane 829. Pressure inside the air space 837 may counteract force applied by the plunger 806 (e.g., when the air space 837 is sealed), such that it may be more difficult to actuate the plunger 806 as the flexible membrane 829 expands and causes air (or other gas) in the air space 837 to be compressed.

In implementations that include a sealed housing 836, release, by a user, of any force on the plunger 806 may enable the flexible membrane 829 to contract, forcing the mixture 835 back through the through-pores 826 and into the lumen 834 and interior space 810 of the syringe (thereby, in some implementations, pushing the plunger 806 back). As the mixture 835 is forced through the through-pores 826, mixing continues, and the mixture 835 may become even more homogenous. Contraction of the flexible membrane 829 is depicted in FIGS. 8E, 8F, and 8G.

As depicted in FIG. 8H-8J, the plunger 806 can again be actuated (e.g., a user of the syringe 804 can again depress the plunger 806, which may have been reset to its initial position, as described above), causing the mixture to again be forced through the through-pores 826. After the flexible membrane 829 is again expanded (e.g., as depicted in FIG. 8J), a user may again release force on the plunger 806, allowing the flexible membrane 829 to again contract (as depicted in FIGS. 8K-8M).

In some implementations, this process of actuating and releasing the plunger 806—such that the mixture is forced back and forth through the through-pores 826—is repeated multiple times, such that the mixture has a desired homogeneity or other characteristic. In some implementations, a desired characteristic may include formation of a homogenous foam (e.g., in implementations in which components of a sclerosant are chemically mixed); in still other implementations, some other characteristic may result.

Whatever the specific desired characteristics are for the mixture 835, when that mixture 835 is ready to be dispensed, the valve 843 of the stopcock 840 can be opened, as depicted in FIG. 8N. In some implementations, such a valve 843 is a cylindrical member with a transverse through-hole 844 that can, in an open configuration (shown in FIG. 8N, with the through-hole 844 shown in longitudinal cross section), be aligned with the inlet 841 and outlet 842 of the stopcock 840, such that a channel fluidly couples the inlet 841 and outlet 842; or, the transverse through-hole 844 can be disposed perpendicularly with respect to the aforementioned channel (shown in FIG. 8M, with the through-hole 844 shown in transverse cross section), such that fluid communication between the inlet 841 and the outlet 842 is prevented. Once the valve 840 is opened, the plunger 806 can again be actuated to force the mixture 835 out of the outlet 842, as depicted in FIGS. 8P and 8Q.

In some implementations, it may be advantageous to create a mixture 835 from a first component 813 and second component 830 (e.g., as illustrated in and described with reference to FIGS. 8A-8M); dispense a portion of the mixture 835 (e.g., as illustrated in and described with reference to FIGS. 8N-8Q); then “reinvigorate” the mixture 835 before continuing to dispense it. In such implementations, to reinvigorate the mixture 835, a user may close the valve 843, and again actuate the plunger 806 as depicted in and described with reference to FIGS. 8A-8M; then open the valve 843 and continue dispensing the mixture 835. Reinvigoration of the mixture 835 may be advantageous when certain desirable parameters of the mixture 835 change over a period of time, and the procedure for which the mixture 835 is created and dispenses exceeds that time. For example, in some implementations, the mixing device 801 may be employed to create a homogenous foamed agent; and over time, the foamed agent may break down (e.g., microbubbles within the foam may collapse or coalesce); and reinvigoration of the mixture 835 may restore desirable qualities of the foam. In some implementations, the first component 713 and second component 730 mix to provide a useful diagnostic or therapeutic mixture.

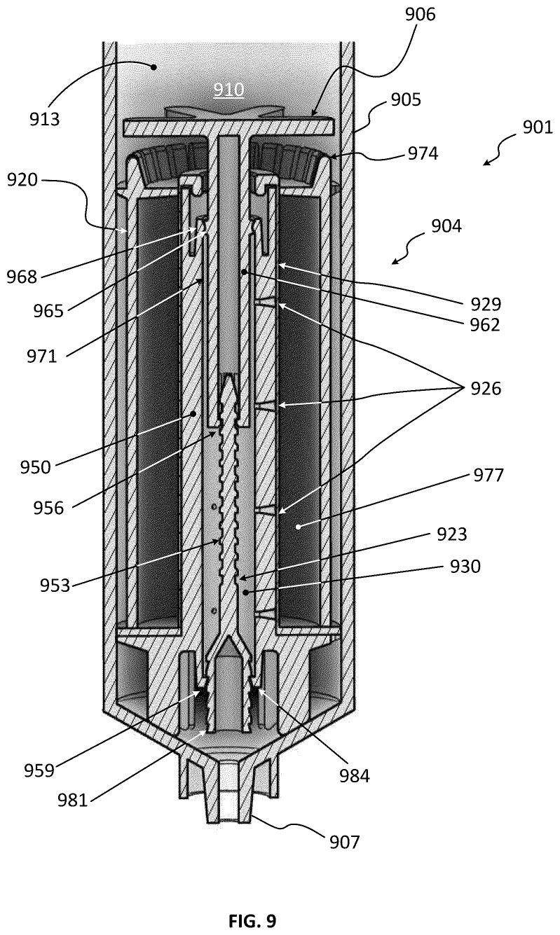

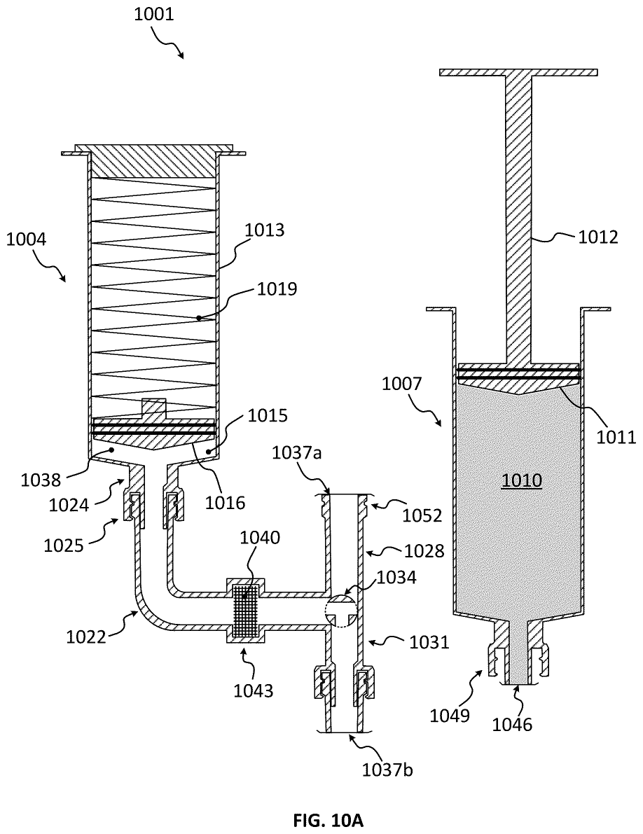

FIG. 9 illustrates another exemplary mixing device 901. In the exemplary mixing device 901, a mixing component 920 may be disposed within the barrel 905 of a syringe 904. As shown, the mixing component 920 may separate the syringe 904 into a first compartment 910 that can hold a first component 913, and a second compartment 923 (e.g., a cylindrical compartment) that can hold a second component 930.

The mixing component 920 may itself include various other components, including, for example, an interior plunger 906 (which can interface with a another plunger (not shown) that may be actuated by a user), a mixing tube 950, and a mixing body 962.

The mixing body 962 includes a circumferential nub 965 that—in an initially neutral position—seals against a corresponding lip 968 of the mixing tube 950. A circumferential channel 971 is formed by a space between the mixing body 962 and an interior lumen of the mixing tube 950. A flexible membrane 929 is disposed around a portion of the mixing tube 950, and through-hole pores 926 extend through a wall of the mixing tube, thereby coupling the circumferential channel 971 and a space between an inner surface of the flexible membrane 929 and an outer surface of the wall forming the interior lumen of the mixing tube 950.

An indexing shaft 953 mechanically interfaces with the mixing body 962 via a first ratcheting mechanism 956. The indexing shaft 953 further interfaces with the mixing tube 950 via a second ratcheting mechanism 959.

In operation, a first component 913 may be disposed in the first compartment 910 (e.g., an interior of the barrel 905 of the syringe), and the second component 930 may be disposed in the second compartment 923. The nub 965 and lip 968 may initially keep the first component 913 and second component 930 separate.

Upon user actuation of a primary plunger (not shown), the interior plunger 906 may be actuated. This actuation (and corresponding vertical translation of the mixing body 962) breaks the seal created by the nub 965 and lip 968, allowing fluid communication between the compartment 910, the channel 971 and the compartment 930—which, in turn, can allow the first component 913 and the second component 923 to mix. Further actuation of the plunger and interior plunger 906 can cause the mixture in the compartment 923 to be forced through the through-holes 926, causing the flexible membrane 929 to be distended.

In some implementations, a region 977 is filled with a gas (e.g., air or some other compressible gas) and sealed, such that it exerts a force against the flexible membrane 929 as the flexible membrane 929 is distended. Spring tabs 974 may exert additional force against the interior plunger 906, such that, upon release of force by a user on the plunger, the flexible membrane 929 is forced back to its original position against a wall of the mixing tube 950, forcing the mixture back into the compartment 930, and forcing the mixing body 962 upward. This action may also advance one or both of the first ratcheting mechanism 956 and the second ratcheting mechanism 959.

Additional force applied by a user on the plunger can again act on the interior plunger 906, forcing it to translate downward and the above-described process to repeat—causing additional mixing of the first component 913 and second component 923, and further causing the resulting mixture to be forced through the through-holes 926, thereby again distending the flexible membrane 929. In this manner, mechanical agitation and mixing of the first component 913 and the second component 930 may be provided by the mixing device 901 in a similar manner as in the mixing device 801—but interior to the barrel 905 of the syringe 904.

The first ratcheting mechanism 956 and second ratcheting mechanism 959 may provide the additional benefit of facilitating a specific number of actuations before the second ratcheting mechanism 959 causes the indexing shaft 953 and its lower seal 981 to be drawn above a corresponding lip 984, such that the mixture can be expelled from the mixing device 901 at the tip 907. In some implementations, by varying the spring force and number of teeth associated with the first ratcheting mechanism 956 and second ratcheting mechanism 959, it may be possible to control a number of actuations of the interior syringe 906, prior to a mixture of the first component 913 and the second component 930 being expelled from the mixing device 901. In such implementations, uniformity of the resulting mixture may be more precisely controlled relative to other implementations that lack features for regulating the amount of mixing.

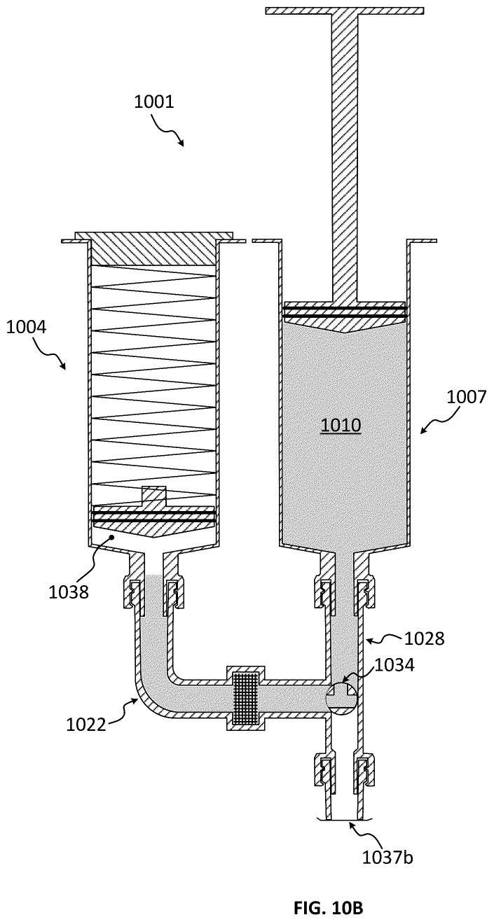

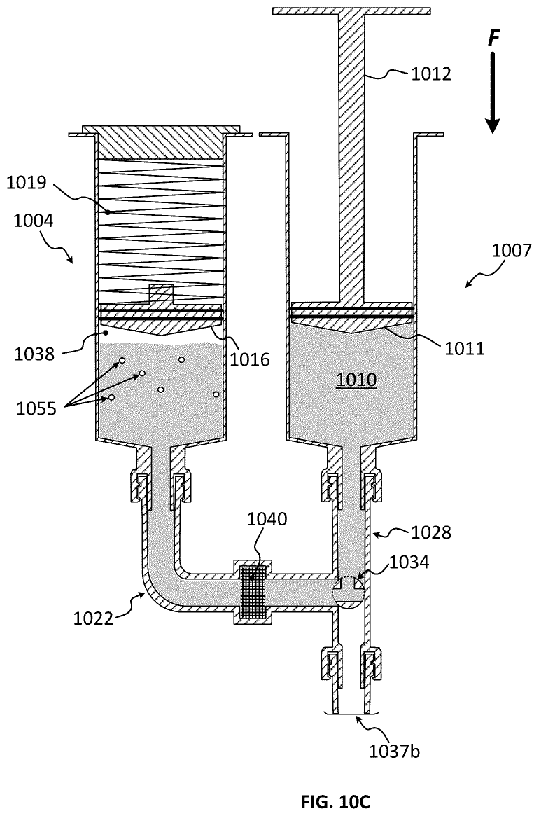

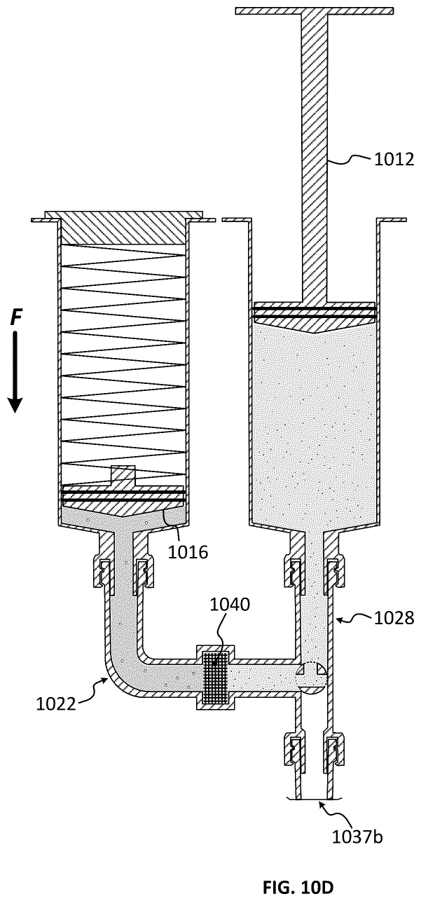

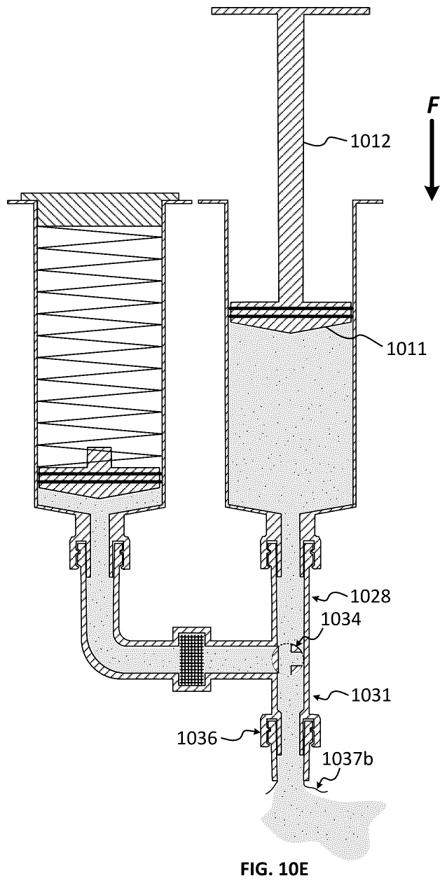

In other implementations, devices may be provided for making a foam on demand—for example, a therapeutic foam for use in delivering therapy to a patient (e.g., sclerotherapy). With reference to FIG. 10A, a mixing kit 1001 may be provided that includes a mixing device 1004 and a syringe 1007 that may contain a foamable therapeutic 1010. The mixing device 1004 may include a syringe body 1013 having in its interior 1015 a plunger 1016 and a spring 1019. The spring 1019 may be metallic, plastic or may include another device, such as an air shock, which is capable of converting kinetic energy into potential energy and back into kinetic energy.

In the implementation shown, the syringe body 1013 is fluidly coupled to a mixing channel 1022 via a mixing tip 1024. The coupling may be implemented with a Luer lock connector 1025, some other coupling, or with an adhesive or molded connection. The mixing channel 1022 is coupled to a supply channel 1028 and a delivery channel 1031. A three-way valve 1034 (e.g., a stopcock) may selectively decouple the supply channel 1028 from either the mixing channel 1022 and delivery channel 1031 (as shown in FIG. 10A); couple the supply channel 1022 to the mixing channel 1022; or couple the supply channel 1029 to the delivery channel 1031.

Removable seals 1037a and 1037b may be provided with the mixing device 1004 to, in some implementations, maintain a sterile environment within the mixing channel 1022, supply channel 1028, delivery channel 1031 and syringe body 1013. In some implementations, the mixing device 1004 is provided with a quantity of gas 1038 (e.g., sterile room air, oxygen, carbon dioxide, etc.) to be mixed with the foamable therapeutic 1010. In some implementations, the gas 1038 is sterile; in other implementations it is not.

A mixing screen or other mixing element 1040 may be provided to introduce turbulence within the mixing channel 1022 when a stream of fluid or gas traverses the same. Multiple (e.g., sequential) mixing elements may be provided in some implementations. For example, two or more screens having mesh openings of approximately 10 μm or 25 μm, or 100 μm or 500 μm may be provided. As another example, screens of different dimensions may be provided (e.g., outer 500 μm mesh screens with a 100 μm mesh screen in the middle). As with other implementations described herein, materials other than screens may be employed to induce turbulence (e.g., sintered or porous elements) and/or break larger bubbles into smaller bubbles as a foam is created.

In some implementations, resistance to fluid flow presented by the mixing element(s) 1040 may be controlled, such that fluid may be rapidly exchanged between the mixing device 1004 and the syringe 1007. For example, a lower mesh size may be used in some implementations with a larger surface area for the mesh itself (and a larger cavity 1043 for retaining the mesh); in other implementations, larger mesh sizes may be employed with less surface area. Numerous variations are contemplated.

The syringe 1007 includes a plunger 1011 and handle 1012 for actuation, and it may also be initially sealed with a seal 1046. The syringe 1007 may also have a Luer lock connector 1049 (or other connector) that is configured to mate with a corresponding connector 1052 on the mixing device 1004.

To use the mixing kit 1001, a user may remove the seals 1037a and 1046 and couple the syringe 1007 to the mixing device 1004 (e.g., by screwing the syringe 1007 onto the mixing device 1004 with corresponding Luer locks 1049 and 1052 (or with other connectors)). In some implementations, the syringe 1007 may be provided with the mixing device 1004 and may already be coupled to the mixing device 1004. In such implementations, removable or breakable seals may isolate the foamable therapeutic 1010 from the various channels 1022, 1028 and 1031 of the mixing device 1004. In other implementations, the syringe 1007 may be supplied by the end user and may be filled on-site with a foamable therapeutic.

FIG. 10B depicts the mixing kit 1001 after seals 1037a and 1046 have been removed, the syringe 1007 has been coupled to the mixing device 1004, the three-way valve 1034 has been adjusted to permit fluid communication between the supply channel 1028 and mixing channel 1022 (but not with the delivery channel 1031), and a small amount of plunging force has been applied to the handle 1012, such that some foamable therapeutic 1010 has been injected into the supply channel 1028 and mixing channel 1022.

FIG. 10C depicts a state in which additional force has been applied to the plunger 1011 (e.g., by user actuation of the handle 1012) and foamable therapeutic 1010 has been injected further into the mixing device 1004. As shown, some gas 1038 remains in the mixing device 1004 initially as gas, as foamable therapeutic 1010 is injected; though some of this gas may begin to dissolve into the foamable therapeutic 1010 (depicted by bubbles 1055). As the foamable therapeutic is injected further into the mixing device 1004, the spring 1019 is compressed—thereby converting the kinetic movement of the foamable therapeutic 1010 into the mixing device 1004 to potential energy. When the force is removed from the handle 1012 and plunger 1011, the potential energy stored in the spring 1019 is converted back to kinetic energy, specifically as the spring force plunges the plunger 1016 to push the foamable therapeutic out of the mixing device 1004 and through the mixing channel 1022 and screen 1040, through the supply channel 1028, and back into syringe 1007 (see FIG. 10D).

In some implementations, spring force of the spring 1019 may be configured to achieve a particular fluid speed or pressure through the mixing element 1040. Specific pressures or speeds of the fluid through the mixing element 1040 may provide sufficient turbulence to cause the foamable therapeutic 1010 to foam (e.g., by causing the gas 1038 to be mixed into the foamable therapeutic in the form of very small bubbles). The mixing element 1040 may cause larger bubbles to be broken down into smaller bubbles; and this mixing and foaming process may be iterative as the foamable therapeutic is forced back and forth through the mixing element 1040.

In some implementations, two advantages of the design just illustrated and described may follow. First, regardless of variability in the speed at which a user actuates the handle 1012 (which may depend on hand strength or on the force intentionally applied by the user), the pressure on the foamable therapeutic by the spring 1019 may be more constant (e.g., related to the energy stored in the spring 1019 and the resistance to flow presented by the mixing element 1040). Thus, even if a particular user with weak hand strength may be less efficient than another user with stronger hand strength in making foam by forcing the foamable therapeutic through mixing element 1040 from the syringe 1007 to the mixing device 1004, the efficiency with which foam is formed in the opposite direction (mixing device 1004 to syringe 1007) may be more uniform. Secondly, operation of the mixing kit 1001 may be possible with only one hand of the user. This can be very advantageous relative to other designs, which may require the user to hold and actuate multiple components (two syringes, for example). With the mixing kit 1001, a user may have another hand free for other aspects of a procedure (e.g., operating an ultrasound probe or attending to other aspects of a therapeutic procedure). In some implementations, this one-handed operation that is possible may allow a procedure to be performed with fewer clinicians and technicians or more quickly and easily.

The process of applying force to the handle 1012 and plunger 1011 may be repeated multiple times to create a foam comprising very small, stable bubbles that, in some implementations, resist coalescence over a long period of time. When a therapeutic foam having desirable properties has been formed, the three-way valve 1034 may be actuated to fluidly couple the syringe 1007, supple channel 1028 and delivery channel 1031; any seal 1037b that is still present may be configured to break upon application of a dispensing force from actuation of the handle 1012 and plunger 1011 (or any seal 1037b may be removed prior to dispensing); and the now-foamed therapeutic may be dispensed (see FIG. 10E). In some implementations, a connector 1036 may be coupled to a line or needle (tip not shown) for application of the foamed therapeutic to a therapy site.

Various references have been made to a foamable therapeutic. In some applications, the foamable therapeutic may be a composition that is suitable for sclerotherapy. Exemplary components may include hypertonic saline, sodium tetradecyl sulfate (STS), polidocanol, and chromated glycerin. One or more foam-stabilizing compounds may also be included. For example, proteins (e.g., albumin), glycerol, or proteoglycans may be added, e.g., to extend the working life of a resulting foam. One or more surfactants may be added, e.g., as wetting agents, emulsifiers, foaming agents, or dispersants—including, for example, polysorbate (e.g., PS-80) or ethanol. In general, formulations may include an aqueous buffer comprising water and one or more salts (e.g., saline, potassium chloride); at least one foam-stabilizing compound (e.g., a protein, such as albumin); at least one surfactant; and a sclerosant (e.g., polidocanol or STS). Other variations are contemplated.

Several implementations have been described with reference to exemplary aspects, but it will be understood by those skilled in the art that various changes may be made, and equivalents may be substituted for elements thereof without departing from the contemplated scope. For example, needles for piercing a membrane of a container are illustrated and described as being concentric around a common axis, but separate needles may be employed. Needle tips or edges may be sharpened. A liquid agent and a biologically compatible gas are described, but two liquid agents having different densities may be employed; alternatively, with appropriate modifications (e.g., additional membranes or films to separate agents) and depending on chemical interactions therebetween, it may be possible to employ solid (e.g., powdered) agents with liquid, gas, or other solid agents. Screens may be differently disposed than described or may have different design parameters; more or fewer screens may be employed; sponges, porous elements, sintered elements, or other structures may be employed in place of screens. Check valves may be differently disposed or constructed than described or illustrated, and more or fewer check valves may be employed, or they may be replaced by manually actuated valves. The syringe may be manually actuated by a human user, machine-actuated (e.g., with a spring, a pneumatic cylinder, an electrical solenoid, etc.), or actuated by a combination of machine and human user (e.g., a human user may release a mechanical catch that releases a spring, or a human user may control a pneumatically or electrically operated mechanism).

Many other variations are possible, and modifications may be made to adapt a particular situation or material to the teachings provided herein without departing from the essential scope thereof. Therefore, it is intended that the scope include all aspects falling within the scope of the appended claims.

What is claimed is:

1. A method of making and providing a therapeutic foam comprising:
providing (i) a syringe and (ii) a mixing device; the syringe having a barrel, a plunger, a tip, and a foamable therapeutic disposed in the barrel; the mixing device having (A) a syringe body and a mixing tip fluidly coupled to an interior of the syringe body, and a plunger and spring disposed within the syringe body; (B) a mixing channel, a supply channel and a delivery channel; wherein one end of the mixing channel is coupled to the mixing tip and one end of the supply channel includes a connector for removably coupling to the syringe; (C) a three-way valve that is configured to selectively couple an opposite end of the mixing channel, an opposite end of the supply channel and the delivery channel to one or more of the others; and (D) a mixing element disposed in the mixing channel;
coupling the syringe to the connector;
actuating the three-way valve to couple the supply channel to the mixing channel, but not to the delivery channel;
plunging the plunger of the syringe to force foamable therapeutic through the supply channel, mixing channel and mixing element and into the mixing device, thereby causing the spring to be compressed;
releasing force on the plunger of the syringe to allow the spring to force the foamable therapeutic back through the mixing channel, mixing element and supply channel and into the syringe, thereby creating a foamed therapeutic;
actuating the three-way valve to couple the supply channel to the delivery channel, but not to the mixing channel; and
plunging the plunger to dispense the foamed therapeutic.

2. The method of claim 1, further comprising repeating the plunging and releasing steps one or more times.

3. The method claim 1, wherein the mixing element comprises a mesh screen characterized by apertures of between about 100 μm and 500 μm.

4. The method claim 1, wherein the mixing element comprises a mesh screen characterized by apertures of between about 10 μm and 25 μm.

5. The method claim 1, wherein the mixing element comprises a sintered or porous material.

6. A kit comprising:
a syringe; and
a mixing device;
the syringe having a barrel, a plunger, a tip, and a foamable therapeutic disposed in the barrel; and the mixing device having (A) a syringe body and a mixing tip fluidly coupled to an interior of the syringe body, and a plunger and spring disposed within the syringe body; (B) a mixing channel, a supply channel and a delivery channel; wherein one end of the mixing channel is coupled to the mixing tip and one end of the supply channel includes a connector for removably coupling to the syringe; (C) a three-way valve that is configured to selectively couple an opposite end of the mixing channel, an opposite end of the supply channel and the delivery channel to one or more of the other; and (D) a mixing element disposed in the mixing channel;
wherein the kit is configured to allow (x) the syringe to be removably coupled to the connector; (y) the three-way valve to be actuated to couple the supply channel to the mixing channel, but not to the delivery channel; (z) the plunger to be plunged to force foamable therapeutic through the supply channel, mixing channel and mixing element and into the mixing device, thereby causing the spring to be compressed; (aa) the plunger to be released, to allow the spring to force the foamable therapeutic back through the mixing channel, mixing element and supply channel and into the syringe, thereby creating a foamed therapeutic; (bb) the three-way valve to be actuated to couple the supply channel to the delivery channel, but not to the mixing channel; and (cc) the plunger to be plunged to dispense the foamed therapeutic.

7. A method of making a therapeutic foam comprising:
providing a syringe having a plunger; a housing coupled to the syringe; and a container; wherein (a) the housing has a first inlet port that couples to the syringe, a second inlet port having a first needle and a second needle, an outlet port, a mixing chamber, a first check valve that permits fluid communication from the second inlet port to the mixing chamber but not from the mixing chamber to the second inlet port, a second check valve that permits fluid communication from the mixing chamber to the outlet port but not from the outlet port to the mixing chamber, and at least one screen disposed between the first check valve and the mixing chamber, or between the second check valve and the mixing chamber, or between the first inlet port and the mixing chamber; and (b) the container comprises a vessel having an opening on one end that is sealed with a pierceable membrane, the vessel having therein a biologically compatible gas and a therapeutic agent that are capable of being combined to form a foam;
coupling the container to the syringe by piercing the pierceable membrane with the second inlet port, such that the first needle extends beyond the therapeutic agent into a region containing the biologically compatible gas and the second needle extends into the therapeutic agent;
deplunging the syringe to draw biologically compatible gas and therapeutic agent from the container into the mixing chamber and syringe to thereby form a therapeutic foam; and
plunging the syringe to expel the therapeutic foam from the housing.

8. The method of claim 7, wherein the first needle and second needle comprise a dual-lumen needle, wherein the second needle is concentrically disposed around the first needle, and wherein the first needle extends beyond the second needle.

9. The method of claim 7, wherein the therapeutic agent is in liquid form in the container.

10. The method of claim 7, wherein the pierceable membrane is a self-healing pierceable membrane.

11. The method of claim 7, wherein the container comprises glass.

12. The method of claim 7, wherein the container comprises a coated material that inhibits diffusion of gases into or out of the container.

13. The method of claim 7, wherein the at least one screen comprises a mesh having openings of about 25 μm.

14. The method of claim 7, wherein the at least one screen comprises a mesh having openings of about 10 μm.

15. The method of claim 7, wherein the container further comprises a pressure-equalization channel that is fluidly coupled to an expandable pressure-equalization chamber and that is configured to couple to a pressure-equalization passage in the housing.

16. The method of claim 15, wherein the pressure-equalization passage is open to an exterior of the housing on one end and comprises a needle at its opposite end, which needle is configured to pierce the pierceable membrane and couple to the pressure-equalization channel when the container is disposed on the housing.

17. The method of claim 16, wherein an interior of the expandable pressure-equalization chamber is isolated from the therapeutic agent and biologically compatible gas in the container.

18. The method of claim 16, wherein the expandable pressure-equalization chamber comprises an expandable balloon structure that is configured to inflate with gas that enters its interior through the one end, the pressure equalization passage and the pressure-equalization channel whenever a negative pressure exists in an interior of the container, such that its inflation and corresponding increase in volume displaces therapeutic agent and biologically compatible gas that has been withdrawn from the container.

19. A mixing and delivery device comprising:
a syringe having a barrel, a plunger, and a tip; the barrel having a sidewall that, with the plunger, defines an interior space; the interior space comprising a first fluid component and being fluidly coupled to a discharge port at the tip
a mixing channel having a channel wall that is characterized by a thickness, that defines an interior volume and that has an outer surface; the mixing channel having an inlet end, an outlet end, and a plurality of through-pores disposed through the thickness to fluidly couple the interior volume and a space adjacent and exterior to the mixing channel; the mixing channel further comprising a flexible membrane that circumferentially surrounds the outer surface and that is sealed to the outer surface at the inlet end and the outlet end; the interior volume comprising a second fluid component;
a seal disposed at the inlet end to initially separate the first fluid component and the second fluid component; and
a stopcock having an inlet, an outlet and a valve, wherein the inlet is coupled to the outlet end, and the valve has an open configuration that facilitates fluid coupling of the inlet and outlet; and a closed configuration that prevents fluid coupling of the inlet and outlet.

20. The mixing and delivery device of claim 19, wherein the seal is a sealing membrane that is configured to rupture when the plunger is depressed, thereby allowing the first fluid component and the second fluid component to mix.

21. The mixing and delivery device of claim 19, wherein the flexible membrane is configured to distend to facilitate transport of fluid from the interior space and interior volume, through the plurality of through-holes, into a mixing space, when the plunger is depressed.

22. The mixing and delivery device of claim 21, wherein the flexible membrane has an elasticity which, when the flexible membrane is in a distended state, exerts a force on fluid in the mixing space causing said fluid to be forced back through the through-holes, into the interior volume, when such exerted force exceeds counterbalancing pressure of the fluid.
